(19) United States
(12) Patent Application Publication
(10) Pub. No.: US 2021/0346011 A1
(43) Pub. Date: Nov. 11, 2021

(54) MENISCAL REPAIR NEEDLE AND MENISCAL REPAIR SYSTEM

(71) Applicant: The First Affiliated Hospital of University of South China, Hengyang (CN)

(72) Inventor: Zhu DAI, Hengyang (CN)

(21) Appl. No.: 17/239,508

(22) Filed: Apr. 23, 2021

(30) Foreign Application Priority Data
May 6, 2020 (CN) 22010370450.5

(51) Int. Cl. A61B 17/06; A61B 17/04

(52) U.S. Cl. CPC A61B 17/06; A61B 17/0491; A61B 17/56; A61B 17/06004; A61B 17/06066

(57) ABSTRACT

A meniscal repair needle, comprising a needle rod, wherein the front section of the needle rod is warped in the shape of an arc and gradually reduces in size to form a needle tip as it extends forward, wherein the needle rod is configured to be a hollow structure, wherein a notch is formed in a front section of the needle rod, and wherein the notch expands inwards from the surface of the needle rod forming a hook portion, wherein an inner cavity of the needle rod extends into the notch, and wherein a hook tip of the hook portion faces the rear of the needle rod, wherein a rear section of the needle rod is connected with a hand-held portion.

### TECHNICAL FIELD

This invention generally relates to the technical field of medical instruments, and more particularly, to a meniscal repair needle and a meniscal repair system.

### BACKGROUND

Meniscus tears are among the most common knee injuries. A meniscectomy is typically required when the injury occurs in a non-vascularized area, and a meniscal repair is typically performed when the injury occurs in a vascularized area. Methods for repairing a torn meniscus typically include an outside-in suturing method, an inside-out suturing method and an all-inside suturing method. Presently, tears of the posterior segment of the meniscus are usually repaired by adopting the all-inside suturing method using special instruments, while tears of the anterior segment and the body of the meniscus are repaired by adopting the outside-in suturing method. When suturing from outside to inside, a lumbar puncture needle is primarily used as a thread-guiding device, which guides the suture thread into the knee joint cavity for suturing. However, it is difficult for a lumbar puncture needle to pass through the meniscus due to its bluntness. Additionally, during meniscal surgery, using a lumbar puncture needle may easily cause a secondary meniscal injury and the cutting-off of the suture thread during puncturing, resulting in thread breakage and poor suturing.

Chinese patent CN202426582U discloses a meniscal repair needle used specially for suturing from outside to inside. Although this repair needle has higher efficiency and is more effective than a conventional lumbar puncture needle, its adaptability and operation efficiency still need to be improved.

Chinese patent CN108523953A discloses a one-needle-one-thread meniscal repair device, which is provided with a guide needle. Under the guidance of the guide needle, accurate entry angle and exit point of the meniscal repair needle are achieved. Moreover, the meniscal repair needle is provided with a groove for receiving the thread, which effectively prevents the thread from being cut off. However, the aforesaid technical solution only solves the technical problems relating to the thread breakage and the accurate control of the needle. The shortcomings such as the inconvenience of use and low operation efficiency still exist.

Chinese patent CN109512478A discloses a meniscal repair device for performing an outside-in suturing, wherein a thread outlet is provided on the side wall of the needle, and the suture thread is pulled out from the thread outlet, which prevents the thread from being cut off by the sharp portion of the needle. When the aforesaid meniscal repair device is used for a single-needle suturing, the thread needs to be pulled out twice using a thread grabber, and then the thread is tied in a knot in the knee joint using a push connector. Its adaptability and operation efficiency still need to be improved.

### SUMMARY

The purpose of the present invention is to provide a meniscal repair needle for outside-in suturing of the torn parts of the anterior segment and the body of the meniscus, which achieves high convenience of use, high operation efficiency and better suturing effect. The present invention also provides a meniscal repair system based on the meniscal repair needle.

To achieve the above purpose, the present invention adopts the following technical solution: a meniscal repair needle comprising a needle rod, wherein the front section of the needle rod is warped in the shape of an arc and gradually becomes smaller to form a needle tip as it extends forward, wherein the needle rod is configured to be a hollow structure, wherein a notch is formed in the front section of the needle rod, and the notch expands inwards from the surface of the needle rod, thus forming a hook portion capable of hooking and pulling the thread at the notch, wherein the notch is communicated with the inner cavity of the needle rod, and the hook tip of the hook portion faces the rear of the needle rod, wherein the rear section of the needle rod is connected with a hand-held portion for allowing a user's hand to grasp, a first fixing portion for fixing one end of the suture thread and a second fixing portion for fixing the other end of the suture thread, wherein when using the meniscal repair needle, the suture thread can be inserted into the inner cavity of the needle rod from the rear end of the needle rod and penetrates out through the notch, and then the two ends of the suture thread are respectively fixed through the first fixing portion and the second fixing portion.

In another aspect of the present invention, the needle rod is further provided with a cylindrical skin distraction device which is sleeved on the needle rod and is capable of moving forwards and backwards along the axial direction of the needle rod.

In another aspect of the present invention, external threads are arranged on the outer peripheral surface of the middle section of the needle rod, internal threads are correspondingly arranged on the inner wall surface of the skin distraction device, and the skin distraction device is in threaded connection with the needle rod.

In another aspect of the present invention, the first fixing portion comprises a first lug having an anti-sliding structure on the surface. When the meniscal repair needle is used, one end of the suture thread is wound on the first lug. Through the arrangement of anti-sliding patterns or convex points, the suture thread can be prevented from slipping off the first lug under the action of an external force, thereby realizing the fixation of the suture thread.

In another aspect of the present invention, the second fixing portion comprises a second lug. When the meniscal repair needle is used, a loop can be formed through tying a knot at the end of the suture thread first, and then the loop is hung on the second lug to realize the fixation of the suture thread.

In another aspect of the present invention, the hand-held portion comprises a handle which is expanded and enlarged compared with the needle rod. The rear end of the needle rod is inserted into the handle to be fixed, and the rear end of the handle is provided with a threading hole communicated with the inner cavity of the needle rod.

In another aspect of the present invention, the first fixing portion and the second fixing portion are the two lugs arranged on the surface of the handle.

In another aspect of the present invention, the present invention also provides a meniscal repair system comprising a meniscal repair needle with any of the above structures. The suture thread is inserted into the inner cavity of the needle rod of the meniscal repair needle. One end of the suture thread extends from the rear end of the needle rod and is fixed on the first fixing portion, and the other end of the suture thread penetrates out from the notch formed in the needle rod and is fixed on the second fixing portion.

In another aspect of the present invention, the handle is fixedly connected to the rear end of the needle rod, and a threading hole communicated with the inner cavity of the needle rod is formed in the rear end of the handle. The first fixing portion 4 and the second fixing portion are respectively the first lug and the second lug. One end of the suture thread extending from the rear end of the needle rod through the threading hole is wound on the first lug and fixed, and the other end of the suture thread penetrating out of the notch forms a loop which is hung on the second lug and fixed.

Compared with the prior art, the present invention has the following advantages:

The meniscal repair needle of the present invention can also realize the meniscal suturing with one needle and one thread. The meniscal repair needle of the present invention has a needle rod in a hollow structure, wherein the suture thread is inserted into the inner cavity of the needle rod from the rear end of the needle rod and penetrates out through the notch, and the front end of the needle rod gradually becomes smaller to form a sharp needle tip. By means of the aforesaid design, the puncturing becomes more convenient and the suture thread is protected from being cut off. It is particularly worth mentioning that the hook portion formed at the notch can hook the suture thread when performing a meniscal repair. Through adopting the aforesaid design, when advancing the needle for the second time, the advancing of the thread becomes unnecessary. Particularly, it is unnecessary to hook the thread 3 using a hook outside the knee joint to prevent the thread fed in along with the first advancing of the needle from being brought out by the second advancing of the needle. Thus, convenient operation and high efficiency are achieved. Additionally, the meniscal repair needle can also easily realize a double-thread suturing. The specific operation is as follows: when advancing the needle for the first time, both ends of the suture thread are fixed through the first fixing portion and the second fixing portion; after the head of the needle passes through the meniscus, pulling the middle section of the suture thread using a thread grabber to form a loop; after withdrawing the needle, advancing the needle for the second time, and then pulling the loop out by using the hook; after tying a knot under the skin, completing the double-thread suturing. The double-thread suturing has high reliability and allows a thinner suture thread to be used. In this way, the needle rod can be made thinner, the damage caused by the puncturing is smaller, and the operation effect is better. Moreover, through arranging the skin distraction device on the needle rod, before the needle is withdrawn after its first advancing, the skin distraction device can be moved forward along the needle rod to stretch the skin, thus exposing the subcutaneous position of the first advancing. This makes the second advancing of the needle convenient and allows the needle to pass through the skin via the puncture opening of the first advancing except the subcutaneous position is different from the previous one. After the surgery, only one puncture hole is left on the skin surface. Compared with the existing technical solution leaving two puncture holes on the skin surface, it can heal faster with fewer scars and is more aesthetically pleasing.

### BRIEF DESCRIPTION OF THE DRAWINGS

FIG. 1 is a conceptual diagram illustrating an example overall structure of the meniscal repair needle of the present invention.

FIG. 2 is a conceptual diagram illustrating an example overall structure of the meniscal repair needle after the skin distraction device moves forward for a certain distance.

FIG. 3 is a conceptual diagram illustrating an example internal structure of the meniscal repair needle shown in FIG. 1.

FIG. 4 is a conceptual diagram illustrating an existing technique of outside-in suturing the meniscus using one needle and one thread, wherein portion A shows a status of the first advancing of the needle, portion B shows a status of the second advancing of the needle, portion C shows a status of withdrawing the needle after the second advancing of the needle, and portion D shows a status of tying a knot under the skin.

FIG. 5 is a conceptual diagram illustrating a conceptual diagram illustrating a technique adopting the meniscal repair needle shown in FIG. 1 to achieve an outside-in meniscus suturing with one needle and one thread, wherein portion A shows a status of the first advancing of the needle, portion B shows a status of the second advancing of the needle, portion C shows a status of withdrawing the needle after the second advancing of the needle, and portion D shows a status of tying a knot under the skin.

FIG. 6 is a conceptual diagram illustrating a technique adopting the meniscal repair needle shown in FIG. 1 to achieve an outside-in double-thread suturing with one needle and one thread, wherein portion A shows a status of the first advancing of the needle, portion B shows a status of the second advancing of the needle, portion C shows a status of withdrawing the needle after the second advancing of the needle, and portion D shows a status of tying a knot under the skin.

In the Figures:

1—Needle Rod, 2—Hand-held Portion, 3—Suture Thread, 4—The First Fixing Portion, 5—The Second Fixing Portion, 6—Skin Distraction Device, 1a—Notch, 1b—Hook Portion, 2a—Threading Hole.

### DETAILED DESCRIPTION

To facilitate the understanding of those skilled in the art, detailed embodiments and figures are combined hereinafter to further elaborate the technical solution of the present invention. The description in the embodiments is not intended to limit the present invention.

In addition, in the present invention, unless being clearly stated, the orientations or positions indicated by the terms such as “front”, “rear”, “inner” and “outer” are based on the orientations or positions shown in the figures. They are merely used for describing and simplifying the description of the present invention, but not indicating or implying that the equipment or element referred to must have a specific orientation, or be constructed and operated in a specific orientation. Thus, they cannot be understood as a limitation of the present invention.

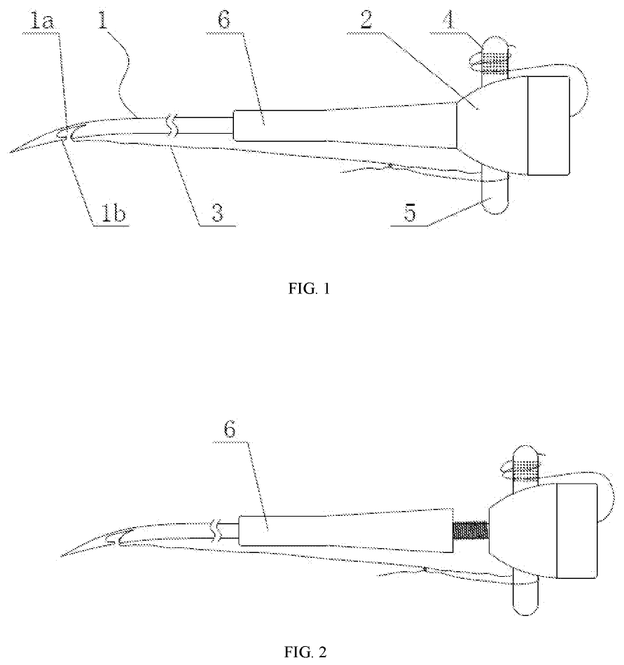

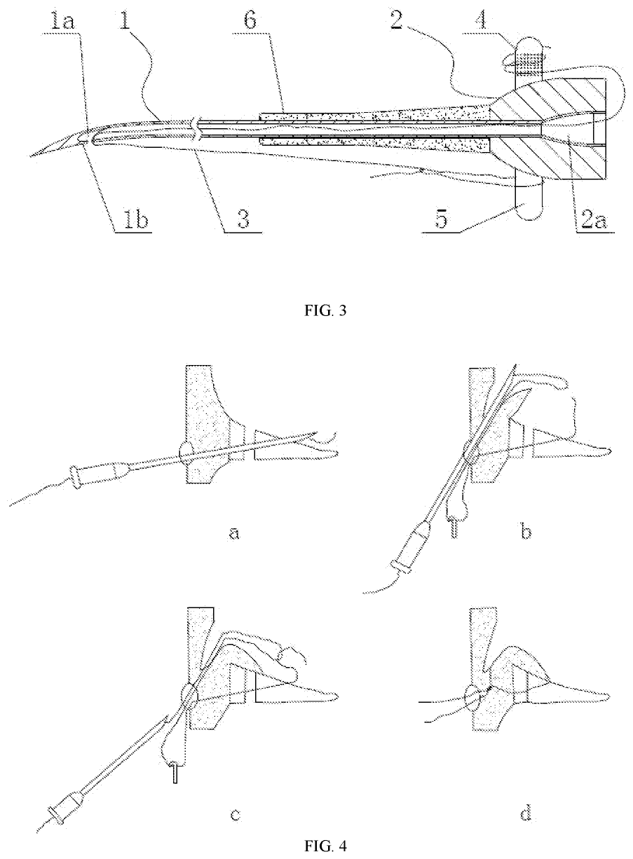

FIGS. 1-3 respectively show a three-dimensional structure and an internal structure of the meniscal repair needle of the present invention. As shown in the figures, the meniscal repair needle of the present invention comprises a needle rod 1, wherein the front section of the needle rod 1 is warped in the shape of an arc and gradually becomes smaller to form a needle tip as it extends forward.

The needle rod 1 is configured to be a hollow structure, wherein a notch 1a is formed in the front section of the needle rod 1, and the notch 1a expands inwards from the surface of the needle rod 1, thus forming a hook portion 1b capable of hooking and pulling the thread at the notch 1a. The notch 1a is communicated with the inner cavity of the needle rod 1, and the hook tip of the hook portion 1b faces the rear of the needle rod 1.

The rear section of the needle rod 1 is connected with a hand-held portion 2 for allowing a user's hand to grasp, a first fixing portion 4 for fixing a first end of the suture thread 3 and a second fixing portion 5 for fixing a second end of the suture thread 3.

When using the meniscal repair needle, the suture thread 3 can be inserted into the inner cavity of the needle rod 1 from the rear end of the needle rod 1 and then penetrates out through the notch 1a, and wherein the two ends of the suture thread 3 can be respectively fixed through the first fixing portion 4 and the second fixing portion 5.

It should be noted that, because the suture thread 3 is frequently used, the meniscal repair needle and the suture thread 3 can be produced and sold separately. The purchased meniscal repair needle can also be clinically used with other existing suture threads.

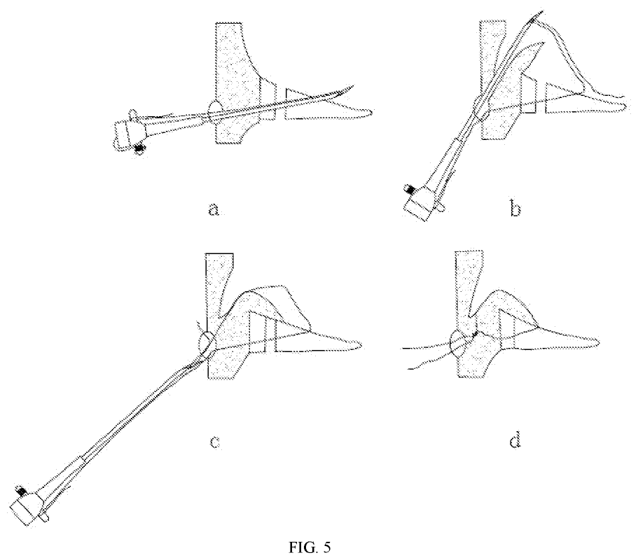

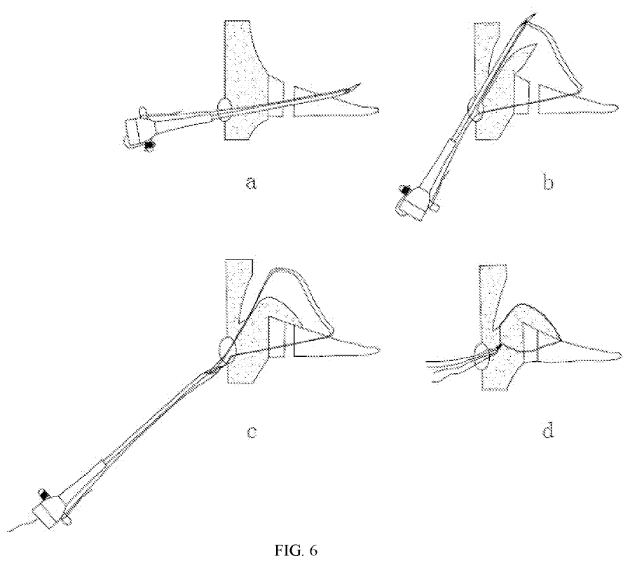

Both the meniscal repair needle shown in FIGS. 1-3 and the conventional outside-in meniscal repair device are capable of conducting meniscal suturing with one needle and one thread. The difference between them is that the meniscal repair needle of the present invention has a needle rod 1 comprising a hollow structure, wherein the suture thread 3 is inserted into the inner cavity of the needle rod 1 from the rear end of the needle rod 1 and penetrates out through the notch 1a, and the front end of the needle rod 1 gradually reduces in size to form a sharp needle tip. By means of the aforesaid design, puncturing becomes more convenient and the suture thread 3 is protected from being cut off. It is particularly worth mentioning that the hook portion 1b formed at the notch 1a can be sued to hook the suture thread 3 when performing a meniscal repair. As shown in FIGS. 4-5, through adopting the aforesaid design, when advancing the needle for a second time, advancing of the thread becomes unnecessary. Particularly, it is unnecessary to hook the thread 3 using a hook outside the knee joint (as the prior one-needle-one-thread suturing does) to prevent the thread fed in along with the first advancing of the needle from being brought out by the second advancing of the needle. Thus, convenient operation and high efficiency are achieved. Additionally, as shown in FIG. 6, the meniscal repair needle can also conduct double-thread suturing. The specific operation is as follows: when advancing the needle for the first time, both ends of the suture thread 3 are fixed through the first fixing portion 4 and the second fixing portion 5; after the tip of the needle passes through the meniscus, pulling the middle section of the suture thread 3 using a thread grabber to form a loop; after withdrawing the needle, advancing the needle for the second time, and then pulling the loop out by using the hook (as shown in portion b of FIG. 6); after tying a knot under the skin, complete the double-thread suturing. The double-thread suturing has high reliability and allows a thinner suture thread 3 to be used. Accordingly, the needle rod 1 can be made thinner, and damage caused by the puncturing using the need rod is therefore smaller.

In addition, the needle rod 1 is further provided with a cylindrical skin distraction device 6 which is sleeved on the needle rod 1 and is capable of moving forwards and backwards along the axial direction of the needle rod 1. Through arranging the skin distraction device 6 on the needle rod 1, before the needle is withdrawn after its first advancing, the skin distraction device 6 can be moved forward along the needle rod 1 to stretch the skin, thus exposing the subcutaneous position of the first advancing. This makes the second advancing of the needle easier and allows the needle to pass through the skin via the puncture opening made by the first advancing although the subcutaneous position is different from the previous one. After the surgery, there will be only one puncture hole left on the skin surface. Compared with the existing technical solution which leaves two puncture holes on the skin surface, the present invention allows faster healing with fewer scars.

More specifically, external threads are arranged on the outer peripheral surface of the middle section of the needle rod 1, internal threads are correspondingly arranged on the inner wall surface of the skin distraction device 6, and the skin distraction device 6 is in threaded connection with the needle rod 1.

As shown in FIGS. 1-3, the first fixing portion 4 comprises a first lug having an anti-sliding structure on the surface. When the meniscal repair needle is used, one end of the suture thread 3 is wound on the first lug. Through the anti-sliding design or the arrangement of convex points, the suture thread 3 can be prevented from slipping off the first lug under the action of an external force, thereby securely fixes the suture thread 3.

The second fixing portion 5 comprises a second lug. When the meniscal repair needle is used, a loop can be formed through tying a knot at the end of the suture thread 3 first (e.g., a Lasso loop), and then the loop is hung on the second lug to securely fix the suture thread 3.

As shown in FIGS. 1-3, The hand-held portion 2 comprises a handle which is expanded and enlarged compared with the needle rod 1. The rear end of the needle rod 1 is inserted into the handle to be fixed, and the rear end of the handle is provided with a threading hole 2a communicated with the inner cavity of the needle rod 1. The two lugs arranged on the surface of the handle are the first fixing portion 4 and the second fixing portion 5.

The present invention also provides a meniscal repair system based on the meniscal repair needle. The difference between them is that the suture thread 3 is pre-inserted into the meniscal repair system. Specifically, the suture thread 3 is pre-inserted into the inner cavity of the needle rod 1 of the meniscal repair needle. More specifically, one end of the suture thread 3 extends from the rear end of the needle rod 1 and is fixed on the first fixing portion 4, and the other end of the suture thread 3 penetrates out from the notch 1a formed in the needle rod 1 and is fixed on the second fixing portion 5.

In the meniscal repair system of the present invention, the hand-held portion 2 adopts a grip handle structure. In addition to the shape shown in the figures which is obviously larger than the needle rod 1, the handle may also be configured to be a shuttle shape, a cylindrical shape, a spindle shape or other shapes. The handle is fixedly connected to the rear end of the needle rod 1, and a threading hole 2a communicated with the inner cavity of the needle rod 1 is formed in the rear end of the handle. The first fixing portion 4 and the second fixing portion 5 are respectively the first lug and the second lug. As shown in the Figures, one end of the suture thread 3 extending from the rear end of the needle rod 1 through the threading hole 2a is wound on the first lug and fixed, and the other end of the suture thread 3 penetrating out of the notch 1a forms a loop which is hung on the second lug and fixed.

To make it easier for those skilled in the art to understand the improvements of the present invention, some of the drawings and descriptions of the present invention have been simplified, and some other elements have been omitted for the sake of clarity. Those skilled in the art shall recognize that these omitted elements may also constitute the content of the present invention.

What is claimed is:

1. A meniscal repair needle, comprising:
a needle rod (1), wherein the front section of the needle rod (1) is warped in the shape of an arc and gradually reduces in size to form a needle tip as it extends forward, wherein the needle rod (1) is configured to be a hollow structure, wherein a notch (1a) is formed in a front section of the needle rod (1), and wherein the notch (1a) expands inwards from the surface of the needle rod (1) forming a hook portion (1b), wherein an inner cavity of the needle rod (1) extends into the notch, and wherein a hook tip of the hook portion (1b) faces the rear of the needle rod (1), wherein a rear section of the needle rod (1) is connected with a hand-held portion (2), wherein the hand-held portion further comprising a first fixing portion (4) for fixing a first end of a suture thread (3) and a second fixing portion (5) for fixing a second end of the suture thread (3), wherein the suture thread (3) is inserted into the inner cavity of the needle rod (1) from the rear end of the needle rod (1) and exits from a front end of the needle rod near the notch (1a), and wherein the first end and second end of the suture thread (3) are respectively fixed through the first fixing portion (4) and the second fixing portion (5).

2. The meniscal repair needle of claim 1, wherein the needle rod (1) is further provided with a cylindrical skin distraction device (6) sleeved on the needle rod (1), wherein the cylindrical skin distraction device is capable of moving forwards and backwards along an axial direction of the needle rod (1).

3. The meniscal repair needle of claim 2, wherein external threads are arranged on the outer peripheral surface of the middle section of the needle rod (1), internal threads are correspondingly arranged on the inner wall surface of the skin distraction device (6), and the skin distraction device (6) is in threaded connection with the needle rod (1).

4. The meniscal repair needle of claim 1, wherein the first fixing portion (4) comprises: a first lug having an anti-sliding structure on the surface, wherein the first end of the suture thread (3) is wound on the first lug.

5. The meniscal repair needle of claim 4, wherein the second fixing portion (5) comprises: a second lug, wherein a loop is formed through tying a knot at the second end of the suture thread (3) first, and then the loop is hung on the second lug.

6. The meniscal repair needle of claim 1, wherein the hand-held portion (2) comprises: a handle, wherein the rear end of the needle rod (1) is inserted into the handle, and wherein a rear end of the handle is provided with a threading hole (2a) connected with the inner cavity of the needle rod (1).

7. The meniscal repair needle of claim 6, wherein the first fixing portion (4) and the second fixing portion (5) are arranged on the surface of the handle.

8. A meniscal repair system, comprising:
a meniscal repair needle of claim 1, wherein the suture thread (3) is inserted into the inner cavity of the needle rod (1) of the meniscal repair needle, wherein first end of the suture thread (3) extends from the rear end of the needle rod (1) and is fixed on the first fixing portion (4), and second end of the suture thread (3) penetrates out from the notch (1a) formed in the needle rod (1) and is fixed on the second fixing portion (5).

9. The meniscal repair system of claim 8, wherein the handle is fixedly connected to the rear end of the needle rod (1), and a threading hole (2a) connected to the inner cavity of the needle rod (1) is formed in the rear end of the handle, wherein the first fixing portion (4) and the second fixing portion (5) are respectively the first lug and the second lug, wherein the first end of the suture thread (3) extending from the rear end of the needle rod (1) through the threading hole (2a) is wound on the first lug, and the second end of the suture thread (3) penetrating out of the notch (1a) forms a loop which is hung on the second lug and fixed.
